(19) United States
(12) Patent Application Publication
(10) Pub. No.: US 2024/0317862 A1
(43) Pub. Date: Sep. 26, 2024

(54) ANTI-B7-H3 MONOCLONAL ANTIBODY AND USE THEREOF

(71) Applicant: AMPSOURCE BIOPHARMA SHANGHAI INC., Shanghai (CN)

(72) Inventors: Cui Wu, Shanghai (CN); Kangyong Zhu, Shanghai (CN); Jiasheng Diao, Shanghai (CN); Qiang Li, Shanghai (CN)

(73) Assignee: AMPSOURCE BIOPHARMA SHANGHAI INC., Shanghai (CN)

(21) Appl. No.: 18/575,642

(22) PCT Filed: Jun. 17, 2022
(86) PCT No.: PCT/CN2022/099344

§ 371 Date: Dec. 29, 2023

(30) Foreign Application Priority Data
Jul. 2, 2021 (CN) 202110752593.7

(51) Int. Cl. C07K 16/28

(52) U.S. Cl. CPC C07K 16/2827; C07K 2317/20; C07K 2317/24; C07K 2317/31; C07K 2317/92

(57) ABSTRACT

Provided in the present invention are an antibody or an antigen-binding fragment thereof against human B7-H3. Further provided are a nucleic acid molecule encoding the antibody, an expression vector and a host cell for expressing the antibody, and a method for producing the antibody. In addition, further provided in the present invention are a pharmaceutical composition containing the antibody or the antigen-binding fragment thereof, and the use thereof in the preparation of a drug for preventing and/or treating various diseases (comprising tumors and autoimmune diseases).

### TECHNICAL FIELD

The present disclosure belongs to the field of therapeutic monoclonal antibodies. More specifically, the present disclosure relates to an antibody or an antigen-binding fragment thereof against human B7-H3 and further relates to the use of the antibody against tumors and autoimmune diseases.

### BACKGROUND

T cell-mediated immune responses play an extremely important role in the antitumor process of organisms, but the activation and proliferation of T cells require not only antigenic signals recognized by T-cell receptors (TCRs), but also signals provided by a second costimulatory molecule. The molecules of the B7 family belong to the immunoglobulin superfamily of costimulatory molecules. More and more studies have shown that the molecules of this family play an important regulatory role in the normal immune function and pathological condition of organisms.

B7-H3 is a member of the B7 family and is a type I transmembrane protein with two different forms of splice variants, among which the extracellular domain of 2IgB7-H3 is composed of two immunoglobulin domains of IgV-IgC while the extracellular domain of 4IgB7-H3 is composed of four immunoglobulin domains of IgV-IgC-IgV-IgC. The mRNA of B7-H3 is widely expressed in a variety of non-lymphoid tissues such as intestines, stomach, lungs, and kidneys, while the protein is not expressed or poorly expressed in normal tissues and cells but is highly expressed in a variety of tumor tissues and thus closely correlated with tumor progression and the survival and prognosis of the patient.

It has been reported clinically that B7-H3 is overexpressed in many types of cancers, including melanoma, colorectal cancer, leukemia, breast cancer, and other tumors (Flem-Karlsen K et al., Curr. Med. Chem., 2020, 27 (24):4062-4086). In addition, it has also been reported in the literature that the expression level of B7-H3 is positively correlated with clinical pathology malignancy in prostate cancer (Roth T J et al., Cancer Res., 2007, 67 (16):7893-7900). Similarly, for glioblastoma multiforme, B7-H3 expression is negatively correlated with survival, and in pancreatic cancer, B7-H3 expression is correlated with lymph node metastasis and pathology progression. Therefore, B7-H3 is considered a new tumor marker and a potential therapeutic target. In addition to its immunomodulatory effect, B7-H3 has intrinsic pro-tumorigenic activity and is correlated with enhanced cell proliferation, migration, invasion, angiogenesis, metastatic ability, and anticancer drug resistance. It has also been found that B7-H3 is capable of modulating key metabolic enzymes and promoting the high glycolysis ability of cancer cells.

Currently, B7-H3 antibodies have been reported in patents such as WO2012147713, WO2015181267, WO2016044383, WO2017180813, WO2020094120, etc. Most of the anti-B7-H3 antibodies are currently in clinical phase I and II, and no antibody drug against B7-H3 has come into the market. Therefore, it is necessary to further develop a B7-H3 antibody with higher activity, high affinity, and high stability, which will provide more drug options for tumor patients.

### SUMMARY

The present disclosure discloses an antibody or an antigen-binding fragment thereof that binds to B7-H3 with high affinity and specificity. The present disclosure further provides a nucleic acid molecule encoding the antibody or the antigen-binding fragment thereof, an expression vector, a host cell, and a method for producing the antibody. The present disclosure further provides a bispecific antibody, a multi-specific antibody and a pharmaceutical composition including the antibody or the antigen-binding fragment thereof. In addition, the present disclosure further provides the use of the anti-B7-H3 antibody or the antigen-binding fragment thereof disclosed in the present disclosure in the preparation of a drug for the treatment of a tumor (alone or in combination with other active agents or therapeutic methods).

In a first aspect, the present disclosure provides an antibody or an antigen-binding fragment thereof that is capable of specifically binding to B7-H3.

The antibody or the antigen-binding fragment thereof includes a heavy chain variable region (VH) including at least one, two or three complementarity-determining regions (CDRs) selected from the group consisting of:
(i) HCDR1 having a sequence as shown in SEQ ID NO: 5 or 11 or having a sequence with one or more amino acid substitutions, deletions or additions (for example, one, two or three substitutions, deletions or additions) relative to any of the above sequences;
(ii) HCDR2 having a sequence as shown in SEQ ID NO: 6, 12, 24 or 26 or having a sequence with one or more amino acid substitutions, deletions or additions (for example, one, two or three substitutions, deletions or additions) relative to any of the above sequences; and
(iii) HCDR3 having a sequence as shown in SEQ ID NO: 7 or 13 or having a sequence with one or more amino acid substitutions, deletions or additions (for example, one, two or three substitutions, deletions or additions) relative to any of the above sequences; and/or the antibody or the antigen-binding fragment thereof includes a light chain variable region (VL) including at least one, two or three CDRs selected from the group consisting of:
(iv) LCDR1 having a sequence as shown in SEQ ID NO: 8 or 14 or having a sequence with one or more amino acid substitutions, deletions or additions (for example, one, two or three substitutions, deletions or additions) relative to any of the above sequences;
(v) LCDR2 having a sequence as shown in SEQ ID NO: 9, 15 or 25 or having a sequence with one or more amino acid substitutions, deletions or additions (for example, one, two or three substitutions, deletions or additions) relative to any of the above sequences; and
(vi) LCDR3 having a sequence as shown in SEQ ID NO: 10 or having a sequence with one or more amino acid substitutions, deletions or additions (for example, one, two or three substitutions, deletions or additions) relative to the above sequence.

In some preferred embodiments, the substitutions in any one of (i) to (vi) are conservative substitutions.

In some preferred embodiments, HCDR1, HCDR2, and HCDR3 included in the heavy chain variable region and/or LCDR1, LCDR2, and LCDR3 included in the light chain variable region are defined by the Kabat or IMGT numbering system. Table 4 in Example 5 illustrates the CDR amino acid sequences of the murine antibodies defined by the Kabat or IMGT numbering system.

In some preferred embodiments, the antibody or the antigen-binding fragment thereof includes three VH variable region CDRs and three VL variable region CDRs selected from the following five groups:
(i) HCDR1, HCDR2, HCDR3, LCDR1, LCDR2, and LCDR3 each having a sequence as shown in SEQ ID NO: 5, 6, 7, 8, 9 or 10 or having a sequence with one or more amino acid substitutions, deletions or additions (for example, one, two or three substitutions, deletions or additions) relative to any of the above sequences;
(ii) HCDR1, HCDR2, HCDR3, LCDR1, LCDR2, and LCDR3 each having a sequence as shown in SEQ ID NO: 5, 24, 7, 8, 25 or 10 or having a sequence with one or more amino acid substitutions, deletions or additions (for example, one, two or three substitutions, deletions or additions) relative to any of the above sequences;
(iii) HCDR1, HCDR2, HCDR3, LCDR1, LCDR2, and LCDR3 each having a sequence as shown in SEQ ID NO: 5, 6, 7, 8, 25 or 10 or having a sequence with one or more amino acid substitutions, deletions or additions (for example, one, two or three substitutions, deletions or additions) relative to any of the above sequences;
(iv) HCDR1, HCDR2, HCDR3, LCDR1, LCDR2, and LCDR3 each having a sequence as shown in SEQ ID NO: 11, 12, 13, 14, 15 or 10 or having a sequence with one or more amino acid substitutions, deletions or additions (for example, one, two or three substitutions, deletions or additions) relative to any of the above sequences; and
(v) HCDR1, HCDR2, HCDR3, LCDR1, LCDR2, and LCDR3 each having a sequence as shown in SEQ ID NO: 11, 26, 13, 14, 15 or 10 or having a sequence with one or more amino acid substitutions, deletions or additions (for example, one, two or three substitutions, deletions or additions) relative to any of the above sequences.

In some embodiments, the antibody or the antigen-binding fragment thereof is murine or chimeric, the heavy chain variable region of the antibody or the antigen-binding fragment thereof includes a heavy chain FR region of murine IgG1, IgG2, IgG3 or a variant thereof, and the light chain variable region of the antibody or the antigen-binding fragment thereof includes a light chain FR region of a murine κ chain, a murine λ chain or a variant thereof. Table 5 in Example 5 illustrates the variable region amino acid sequences of the preferred murine antibodies.

In some preferred embodiments, the antibody or the antigen-binding fragment thereof includes VH and VL sequences selected from the following two groups:
(i) a VH domain including an amino acid sequence as shown in SEQ ID NO: 1 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain including an amino acid sequence as shown in SEQ ID NO: 2 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and
(ii) a VH domain including an amino acid sequence as shown in SEQ ID NO: 3 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain including an amino acid sequence as shown in SEQ ID NO: 4 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence.

In some embodiments, the antibody or the antigen-binding fragment thereof is humanized. Example 5 gives the basic flow of the humanization strategy, and Table 5 illustrates the variable region amino acid sequences of the preferred humanized antibodies.

In some preferred embodiments, the humanized antibody or the antigen-binding fragment thereof includes VH and VL sequences selected from the following:
(i) a VH domain including an amino acid sequence as shown in SEQ ID NO: 16 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain including an amino acid sequence as shown in SEQ ID NO: 17 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence;
(ii) a VH domain including an amino acid sequence as shown in SEQ ID NO: 18 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain including an amino acid sequence as shown in SEQ ID NO: 19 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence;
(iii) a VH domain including an amino acid sequence as shown in SEQ ID NO: 20 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain including an amino acid sequence as shown in SEQ ID NO: 21 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and
(iv) a VH domain including an amino acid sequence as shown in SEQ ID NO: 22 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain including an amino acid sequence as shown in SEQ ID NO: 23 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence.

In some preferred embodiments, the antibody or the antigen-binding fragment thereof disclosed in the present disclosure further includes a constant region sequence derived from a mammalian (for example, a mouse or a human) immunoglobulin or a variant thereof, and the variant has one or more substitutions, deletions or additions relative to a sequence from which the variant is derived. In some preferred embodiments, the variant has one or more conservative substitutions relative to a sequence from which the variant is derived. In some embodiments, an anti-B7-H3 antibody molecule has a heavy chain constant region (Fc) selected from, for example, heavy chain constant regions of IgG1, IgG2, IgG3, IgG4, IgM, IgA1, IgA2, IgD and IgE, particularly from, for example, heavy chain constant regions of IgG1, IgG2, IgG3 and IgG4, and more particularly from a heavy chain constant region of IgG1, IgG2 or IgG4 (for example, human IgG1, IgG2 or IgG4). In some embodiments, the anti-B7-H3 antibody molecule has a light chain constant region selected from, for example, κ or λ light chain constant regions, preferably a κ light chain constant region (for example, a human κ light chain).

In some embodiments, the constant region is changed, for example, mutated, to modify properties of the anti-B7-H3 antibody molecule (for example, to change one or more of the following properties: Fc receptor (FcR) binding, antibody glycosylation, an effector cell function or a complement function). At least one amino acid residue in the constant region of the antibody may be substituted with different residues to generate a functional change, for example, a change in the affinity of the antibody to an effector ligand (for example, FcR or complement C1q), thereby changing (for example, enhancing, decreasing or eliminating) an effector function. The method for substituting amino acid residues in the Fc region of the antibody to change the effector function is known in the art (see, for example, EP388,151A1, U.S. Pat. No. 564,8260 and U.S. Pat. No. 562,4821). The Fc region of the antibody mediates several important effector functions, such as ADCC, phagocytosis, CDC and etc. In some cases, such effector functions are necessary for therapeutic antibodies; but in other cases, such effector functions may be unnecessary or even harmful, which depends on intended purposes. Therefore, in some embodiments, the antibody or the antigen-binding fragment thereof disclosed in the present disclosure can decrease or even eliminate effector functions (for example, ADCC and/or CDC activity). The amino acid mutation in human IgG4 that stabilizes the structure of the antibody, such as S228P (in the EU numbering scheme, which is S241P in the Kabat numbering scheme), is also considered.

In some exemplary embodiments, the antibody or the antigen-binding fragment thereof disclosed in the present disclosure includes a variant of a heavy chain constant region of human IgG, and the variant has at least one of the following substitutions relative to a wild-type sequence from which the variant is derived: Ser228Pro, Leu234Ala, Leu235Ala, Gly237Ala, M252Y, S254T, T256E, Asp265Ala, Asn297Ala, Pro329Ala, Pro331Ser, Asp356Glu, Leu358Met and M428L (the above-mentioned amino acid positions are positions defined by the EU numbering system, Edelman G M et al., Proc. Natl. Acad. U.S.A., 63, 78-85 (1969). PMID: 5257969).

In some exemplary embodiments, the antibody or the antigen-binding fragment thereof disclosed in the present disclosure includes a variant of a heavy chain constant region of human IgG2, and the variant has the following substitution relative to a wild-type sequence from which the variant is derived: Pro331Ser (a position defined by the EU numbering system). In such embodiments, the antibody or the antigen-binding fragment thereof disclosed in the present disclosure has eliminated the ADCC activity.

In some exemplary embodiments, the antibody or the antigen-binding fragment thereof disclosed in the present disclosure includes a variant of a heavy chain constant region of human IgG4, and the variant has the following substitution relative to a wild-type sequence from which the variant is derived: Ser228Pro (a position defined by the EU numbering system). In such embodiments, the antibody or the antigen-binding fragment thereof disclosed in the present disclosure has a stable structure, can reduce Fab-arm exchange, and does not readily form an incomplete antibody.

In some preferred embodiments, the antibody or the antigen-binding fragment thereof disclosed in the present disclosure is a chimeric antibody or a humanized antibody. In some preferred embodiments, the antibody or the antigen-binding fragment thereof disclosed in the present disclosure is selected from scFv, Fab, Fab′, (Fab′)2, an Fv fragment, a diabody, a bispecific antibody or a multispecific antibody.

The antibody or the antigen-binding fragment thereof disclosed in the present disclosure has high specificity and affinity to B7-H3 (particularly human B7-H3). In some preferred embodiments, the antibody or the antigen-binding fragment thereof disclosed in the present disclosure is capable of binding B7-H3 (particularly human B7-H3) at a KD of about 1 nM or less.

In a second aspect, the present disclosure discloses a nucleotide sequence encoding the anti-B7-H3 antibody or the antigen-binding fragment thereof disclosed in the present disclosure. In some embodiments, the nucleotide sequence encoding the anti-B7-H3 antibody molecule has an optimal codon. For example, the feature of the present disclosure is to separately encode a first nucleic acid and a second nucleic acid of the heavy chain variable region and the light chain variable region of the anti-B7-H3 antibody molecule, and the antibody molecule is selected from any one of: mAb152, mAb272, AB125, AB126, AB127, AB128 or a sequence substantially identical thereto.

For example, the nucleic acid may include a nucleotide sequence of AB125, AB126, AB127 or AB128 or a sequence substantially identical thereto (for example, a sequence having at least about 85%, 90%, 95%, 99% or higher similarity to any of the above sequences, a sequence having one or more nucleotide substitutions (for example, conservative substitutions) relative to any of the above sequences, or a sequence that differ from any of the above sequences by no more than 3, 6, 15, 30 or 45 nucleotides).

In a third aspect, the present disclosure provides a vector (for example, a cloning vector or an expression vector) which includes the separated nucleic acid molecule disclosed in the present disclosure. In some preferred embodiments, the vector disclosed in the present disclosure is, for example, a plasmid, a cosmid or a bacteriophage. In some preferred embodiments, the vector is capable of expressing the antibody or the antigen-binding fragment thereof disclosed in the present disclosure in a subject (for example, a mammal such as a human).

In a fourth aspect, the present disclosure provides a host cell including the separated nucleic acid molecule disclosed in the present disclosure or the vector disclosed in the present disclosure. The host cell may be a eukaryotic cell (for example, a mammalian cell, an insect cell or a yeast cell) or a prokaryotic cell (for example, Escherichia coli). Suitable eukaryotic cells include, but are not limited to, NS0 cells, Vero cells, Hela cells, COS cells, CHO cells, HEK293 cells, BHK cells and MDCKII cells. Suitable insect cells include, but are not limited to, Sf9 cells. In some preferred embodiments, the host cell disclosed in the present disclosure is a mammalian cell, for example, a CHO cell (for example, CHO-K1, CHO-S, CHO DXB11 or CHO DG44).

In a fifth aspect, the present disclosure discloses a pharmaceutical composition including the anti-B7-H3 antibody or the antigen-binding fragment thereof described in the present disclosure and a pharmaceutically acceptable carrier and/or excipient and/or stabilizer.

In some preferred embodiments, the pharmaceutical composition may further include an additional pharmaceutically active agent. In some preferred embodiments, the additional pharmaceutically active agent is a drug with antitumor activity.

In some preferred embodiments, in the pharmaceutical composition, the antibody or the antigen-binding fragment thereof disclosed in the present disclosure and the additional pharmaceutically active agent are provided as separate components or as components of the same composition. Therefore, the antibody or the antigen-binding fragment thereof disclosed in the present disclosure and the additional pharmaceutically active agent may be administered simultaneously, separately or sequentially.

In a sixth aspect, the present disclosure further provides a method for preparing the antibody or the antigen-binding fragment thereof disclosed in the present disclosure. The method includes: (a) obtaining a gene of the antibody or the antigen-binding fragment thereof to construct an expression vector of the antibody or the antigen-binding fragment thereof; (b) transfecting the expression vector into a host cell by a genetic engineering method; (c) culturing the host cell under conditions that allow the antibody or the antigen-binding fragment thereof to be generated; and (d) separating and purifying the generated antibody or the antigen-binding fragment thereof.

The expression vector in step (a) is one or more selected from plasmids, bacteria and viruses, and preferably, the expression vector is pcDNA3.1.

The host cell into which the constructed vector is transfected by the genetic engineering method in step (b) includes a prokaryotic cell, a yeast or a mammalian cell, such as a CHO cell, an NS0 cell or another mammalian cell, and preferably, is a CHO cell.

The antibody or the antigen-binding fragment thereof is separated and purified in step (d) by a conventional immunoglobulin purification method including protein A affinity chromatography and ion exchange, hydrophobic interaction chromatography or molecular sieve.

In a seventh aspect, the present disclosure relates to the use of the antibody or the antigen-binding fragment thereof disclosed in the present disclosure in the preparation of a drug, and the drug is used for preparing a drug or a preparation for the prevention and/or treatment of a tumor.

In some embodiments, the tumor expresses or overexpresses B7-H3. In some embodiments, the tumor is selected from a solid tumor or a hematological tumor (for example, leukemia, lymphoma or myeloma). Specific examples of the tumor include, but are not limited to, lung cancer (for example, lung adenocarcinoma or non-small cell lung cancer (NSCLC)), melanoma (for example, advanced melanoma), renal cancer (for example, renal cell carcinoma), liver cancer (for example, hepatocellular carcinoma), myeloma (for example, multiple myeloma), osteosarcoma, prostate cancer, bladder cancer, urethral cancer, breast cancer, ovarian carcinoma, colorectal cancer, pancreatic cancer, head and neck cancer (for example, head and neck squamous-cell carcinoma (HNSCC)), gastric-esophageal cancer (for example, esophageal squamous-cell carcinoma), mesothelioma, nasopharyngeal cancer, thyroid cancer, cervical cancer, neuroblastoma, glioma, diffuse large B-cell lymphoma, T-cell lymphoma, B-cell lymphoma, non-Hodgkin's lymphoma, myeloid leukemia, chronic lymphocytic leukemia, and acute lymphocytic leukemia.

In an eighth aspect, the present disclosure provides a bispecific molecule including the antibody or the antigen-binding fragment thereof disclosed in the present disclosure. For example, the above-mentioned B7-H3 antibody may be functionally linked to an antibody or an antibody fragment thereof having another antigen binding characteristic to form a bispecific antibody.

In a ninth aspect, the present disclosure provides a method for treating (for example, suppressing and/or delaying) a tumor. The method includes: administering to a subject the anti-B7-H3 antibody described herein or the antigen-binding fragment thereof, for example, a therapeutically effective amount of the anti-B7-H3 antibody or the antigen-binding fragment thereof, alone or in combination with one or more active agents or procedures.

The anti-B7-H3 antibody or the antigen-binding fragment thereof prepared in the present disclosure has high binding affinity to B7-H3 and is highly specific. Data from in vitro biological studies show that the antibody or the antigen-binding fragment thereof prepared in the present disclosure has strong binding activity to target cells. Meanwhile, the antibody disclosed in the present disclosure has an extremely high degree of humanization so that the antibody can be safely administered to a human subject without eliciting an immunogenic response. Further, the antibody disclosed in the present disclosure is expressed in CHO cells and has the advantages of high yield, high activity, simple purification process, and low production cost. Therefore, the antibody of the present disclosure has significant clinical value.

### Abbreviations and Term Definitions

The following abbreviations are used herein:
CDR Complementarity-determining region in a variable region of an immunoglobulin
FR Framework region of an antibody: amino acid residues excluding CDR residues in a variable region of the antibody
IgG Immunoglobulin G
IMGT Numbering system based on the international ImMunoGeneTics information System® (IMGT) initiated by Lefranc et al., referring to Lefranc et al., Dev. Comparat. Immunol., 27:55-77, 2003
mAb Monoclonal antibody
EC₅₀ A concentration at which 50% efficacy or binding is generated
IC₅₀ A concentration at which 50% inhibition is generated
ELISA Enzyme-linked immunosorbent assay
PCR Polymerase chain reaction
HRP Horseradish peroxidase
IL-2 Interleukin 2
K_{D} Equilibrium dissociation constant
ka Association rate constant
kd Dissociation rate constant

In the present disclosure, unless otherwise specified, the scientific and technical terms used herein have meanings generally understood by those skilled in the art. Moreover, the operation steps of cell culture, biochemistry, nucleic acid chemistry and immunology laboratories employed herein are all conventional steps widely used in the corresponding field. Meanwhile, for a better understanding of the present disclosure, the definitions and explanations of related terms are provided below.

The term “EU numbering system (or EU numbering scheme)”: EU refers to the first human IgG1 immunoglobulin, designated Eu, separated and purified by Gerald M Edelman et al., at the end of the 1960s (1968-1969), whose amino acid sequence was determined and numbered (Edelman G M et al., 1969, Proc. Natl. Acad. U.S.A., 63:78-85). The heavy chain constant regions of other immunoglobulins are aligned with Eu for amino acid sequence comparison, and the corresponding amino acid position is the Eu number. The EU numbering system is mainly directed to the immunoglobulin heavy chain constant region, including CH1, CH2, CH3 and hinge regions.

The term “Kabat numbering system (or Kabat numbering scheme)”: In 1979, Kabat et al., first proposed a standardized human immunoglobulin variable region numbering scheme (Kabat E A, Wu T T, Bilofsky H, “Sequences of Immunoglobulin Chains: Tabulation and Analysis of Amino Acid Sequences of Precursors, V-regions, C-regions, J-Chain and β₂-Microglobulins”. 1979. Department of Health, Education, and Welfare, Public Health Service, National Institutes of Health). In Sequences of Proteins of Immunological Interest (Kabat E A, Wu T T, Perry H M, Gottesman K S, Foeller C. 1991. Sequences of Proteins of Immunological Interest (5th edition), Bethesda, MD: U.S. Department of Health and Human Services, National Institutes for Health), Kabat et al., aligned and numbered the amino acid sequences of antibody light and heavy chains. They found that such sequences to be analyzed exhibit variable lengths and that deleted, omitted and inserted amino acids or amino acid fragments may only be present at specific positions. Interestingly, the insertion points are mostly located inside the CDR, but may also be present at certain positions in the framework region. In the Kabat numbering scheme, the light chain variable regions are numbered to position 109, the heavy chain variable regions are numbered to position 113, and the inserted amino acids of the light and heavy chains are identified and annotated by letters (for example, 27a, 27b, . . . ). All Lambda light chains do not include the residue at position 10, whereas Lambda and Kappa light chains are encoded by two different genes on different chromosomes. Lambda and Kappa light chains can be distinguished by differences in their constant region amino acid sequences. Unlike the EU numbering system which is only directed for the heavy chain constant region, the numbering range of the Kabat numbering system covers the full-length immunoglobulin sequences, including the variable and constant regions of the immunoglobulin light and heavy chains.

The term “binding” defines the affinity interaction between a specific epitope on an antigen and its corresponding antibody and is generally understood as “specific recognition”. “Specific recognition” means that the antibody disclosed in the present disclosure does not or substantially does not cross-react with any polypeptide other than a target antigen. The degree of specificity may be determined by immunological techniques, including, but not limited to, immunoblotting, immunoaffinity chromatography, and flow cytometry. In the present disclosure, the specific recognition is preferably determined by flow cytometry, and the criteria for the specific recognition in particular cases can be judged by those of ordinary skill in the art according to the general knowledge of the art which he/she knows.

The term “antigen” is a foreign matter capable of eliciting antibody production by the organism itself or by humans, and is any substance capable of triggering an immune response, such as bacteria and viruses. The foreign antigen molecules are recognized and processed by B cells or antigen-presenting cells (for example, macrophages, dendritic cells, endothelial cells and B cells) and bind to a major histocompatibility complex (for example, MHC II molecules) to form a complex which reactivates T cells and then induces continuous immune responses.

The term “antibody” generally refers to a protein-binding molecule having immunoglobulin functions. Typical examples of the antibody are immunoglobulins and derivatives or functional fragments thereof as long as they exhibit the required binding specificity. Techniques for preparing the antibody are well known in the art. The “antibody” includes different types of natural immunoglobulins (for example, IgA, IgG, IgM, IgD and IgE) and subtypes (for example, IgG1, IgG2, IgA1 and IgA2). The “antibody” further includes non-natural immunoglobulins which include, for example, a single-chain antibody, a chimeric antibody (for example, a humanized murine antibody), and a heteroconjugate antibody (for example, a bispecific antibody) as well as antigen-binding fragments thereof (for example, Fab′, F(ab′)₂, Fab, Fv and rIgG). Reference may also be made to, for example, Pierce Catalog and Handbook, 1994-1995, (Pierce Chemical Co., Rockford, Ill); and Kuby, J, Immunology (3rd edition), WH Freeman&Co., New York, 1997. The antibody can bind to one antigen to be “monospecific”, bind to two different antigens to be “bispecific” or bind to more than one different antigen to be “multispecific”. The antibody may be univalent, bivalent or multivalent, that is, the antibody may bind to one, two or multiple antigen molecules at one time. The antibody “univalently” binds to a specific protein, that is, one molecule of the antibody binds to only one molecule of a protein, but the antibody may also bind to different proteins. When the antibody only binds to each molecule of two different proteins, the antibody “univalently” binds to each protein, and the antibody “bispecifically” and “univalently” binds to each of the two different proteins. The antibody may be “monomeric”, that is, such an antibody includes a single polypeptide chain. The antibody may include multiple polypeptide chains (“multimeric”) or may include two (“dimeric”), three (“trimeric”) or four (“tetrameric”) polypeptide chains. If the antibody is multimeric, the antibody may be a homomultimer, that is, the antibody includes more than one molecule of only one polypeptide chain, including a homodimer, a homotrimer or a homotetramer. Alternatively, the multimeric antibody may be a heteromultimer, that is, the antibody includes more than one different polypeptide chain, including a heterodimer, a heterotrimer or a heterotetramer.

The term “monoclonal antibody (mAb)” refers to an antibody obtained from a substantially homogeneous antibody population, for example, the population includes the same individual antibodies except for possibly small amounts of mutations such as natural mutations. Therefore, the attributive “monoclonal” indicates that the antibody is characterized as a mixture that is not a discrete antibody. The monoclonal antibody is produced by methods known to those skilled in the art, for example, by fusing a myeloma cell and an immune spleen cell to create a hybrid cell that can produce an antibody. Through the synthesis using hybridomas, the contamination with other immunoglobulins is prevented. The monoclonal antibody may be obtained by, for example, a recombination technology, a phage display technology, a synthesis technology or other existing technologies.

The term “intact antibody” refers to an antibody composed of two antibody heavy chains and two antibody light chains. The “intact antibody heavy chain” is composed of, from the N-terminus to the C-terminus, an antibody heavy chain variable domain (VH), an antibody heavy chain constant domain 1 (CH1), an antibody hinge region (HR), an antibody heavy chain constant domain 2 (CH2) and an antibody heavy chain constant domain 3 (CH3), abbreviated as VH-CH1-HR-CH2-CH3, and optionally includes an antibody heavy chain constant domain 4 (CH4) in the case of an antibody of an IgE subtype. Preferably, the “intact antibody heavy chain” is a polypeptide composed of, from the N-terminus to the C-terminus, one VH, CH1, HR, CH2 and CH3. The “intact antibody heavy chain” is a polypeptide composed of, from the N-terminus to the C-terminus, an antibody light chain variable domain (VL) and an antibody light chain constant domain (CL), abbreviated as VL-CL. The antibody light chain constant domain (CL) may be κ (kappa) or λ (lambda). The intact antibody chains are linked to each other by an inter-peptide disulfide bond between the CL domain and the CH1 domain (that is, between the light chain and the heavy chain) and an inter-peptide disulfide bond between the hinge regions of the intact antibody heavy chains. Typical examples of the intact antibody are natural antibodies such as IgG (for example, IgG1 and IgG2), IgM, IgA, IgD and IgE.

The term “antibody fragment” and “antigen-binding fragment” refers to an antigen-binding fragment of the antibody that retains a specific binding ability to an antigen, as well as antibody analogs. It generally includes at least part of an antigen-binding region or a variable region of a parental antibody. The antibody fragment retains at least part of the binding specificity of the parental antibody. Generally, when the activity is represented in moles (K_{D}), the antibody fragment retains at least 10% of parental binding activity. Preferably, the antibody fragment retains at least 20%, 50%, 70%, 80%, 90%, 95% or 100% of the binding affinity of the parental antibody to a target. The antibody fragments include, but are not limited to, Fab fragments, Fab′ fragments, F(ab′)₂ fragments, Fv fragments, Fd fragments, complementarity-determining region (CDR) fragments, disulfide-stabilized variable fragments (dsFv); linear antibodies, single-chain antibodies (for example, scFv monoclonal antibodies), unibodies (technology from Genmab), bivalent single-chain antibodies, single-chain phage antibodies, single domain antibodies (for example, VH domain antibodies), domain antibodies (technology from Domantis), nanobodies (technology from Ablynx); multispecific antibodies formed from antibody fragments (for example, triabodies and tetrabodies); and engineered antibodies such as chimeric antibodies (for example, humanized mouse antibodies) and heteroconjugate antibodies. These antibody fragments can be obtained using any conventional technologies known to those skilled in the art, and the utility of these fragments can be screened in the same way as the intact antibody.

The term “single-chain Fv antibody (or scFv antibody)” refers to an antibody fragment including the VH and VL domains of an antibody. It is a fusion protein of the variable regions of the heavy (VH) and light (VL) chains connected with a linker. The linker enables these two domains to be cross-linked to form an antigen-binding site, and the sequence of the linker generally consists of a flexible peptide, for example, but not limited to, G₂(GGGGS)₃. The size of scFv is generally ⅙ of an intact antibody. The single-chain antibody is preferably an amino acid chain sequence encoded by a nucleotide chain. For the review of scFv, reference may be made to Pluckthun A, 1994. “Antibodies from Escherichia coli”, in The Pharmacology of Monoclonal Antibodies, Vol. 113, Rosenberg M and Moore G P (EDs.), Springer-Verlag, New York, pp 269-315. Reference may also be made to International Patent Application Publication No. WO 88/01649 and U.S. Pat. Nos. 4,946,778 and 5,260,203.

The term “VL domain” refers to an amino-terminal variable domain of an immunoglobulin light chain.

The term “VH domain” refers to an amino-terminal variable domain of an immunoglobulin heavy chain.

The term “hinge region” includes a portion of a heavy chain molecule that links a CH1 domain to a CH2 domain. The hinge region includes about 25 residues and is flexible so that two N-terminal antigen-binding regions freely move. The hinge region may be divided into three different domains: upper, intermediate, and lower hinge domains (Roux K H et al., J. Immunol., 161:4083-4090).

The term “Fab fragment” is composed of a CH1 and a variable domain of a heavy chain and a light chain. The heavy chain of the Fab molecule cannot form a disulfide bond with another heavy chain molecule. The size of “Fab antibody” is ⅓ of an intact antibody, and “Fab antibody” includes only one antigen-binding site.

The term “Fab′ fragment” includes a light chain, a VH domain and a CH1 domain of a heavy chain, and a constant region between CH1 and CH2 domains.

The term “F(ab′)₂ fragment” includes two light chains, VH domains and CH1 domains of two heavy chains, and constant regions between CH1 and CH2 domains, so that an inter-chain disulfide bond is formed between the two heavy chains. Therefore, the F(ab′)₂ fragment is composed of two Fab′ fragments held together by the disulfide bond between the two heavy chains.

The term “Fd fragment” is composed of a CH1 and a variable region of a heavy chain and is the heavy chain of a Fab fragment with the light chain removed.

The term “Fv region” includes variable regions from the heavy chain and the light chain but lacks the constant regions and is the minimum fragment including a complete antigen recognition and binding site.

The term “disulfide-stabilized variable fragment (dsFv)” introduces one cysteine mutation site into VH and VL regions, respectively to form a disulfide bond between the VH and VL regions to achieve structural stability. The term “disulfide bond” includes a covalent bond formed between two sulfur atoms. The amino acid cysteine includes a sulfanyl group which can form a disulfide bond or a bridge with a second sulfanyl group. In most of naturally-occurring IgG molecules, the CH1 and CK regions are linked by a disulfide bond, and two heavy chains are linked by two disulfide bonds at positions 239 and 242 according to the Kabat numbering system (position 226 or 229 in the EU numbering system).

The term “heavy chain constant region” includes an amino acid sequence from the immunoglobulin heavy chain. The polypeptide including the heavy chain constant region includes at least one of: a CHI domain, a hinge domain (for example, an upper hinge region, an intermediate hinge region and/or a lower hinge region), a CH2 domain, a CH3 domain or a variant or fragment thereof. For example, the antigen-binding polypeptide used in the present application may include a polypeptide chain having a CHI domain; a polypeptide having a CHI domain, at least part of a hinge domain, and a CH2 domain; a polypeptide chain having a CHI domain and a CH3 domain; a polypeptide chain having a CHI domain, at least part of a hinge domain, and a CH3 domain; or a polypeptide chain having a CHI domain, at least part of a hinge domain, a CH2 domain, and a CH3 domain. In another embodiment, the polypeptide of the present application includes a polypeptide chain having a CH3 domain. In addition, the antibody used in the present application may lack at least part of a CH2 domain (for example, all or part of a CH2 domain). As described above, it is appreciated by those of ordinary skill in the art that heavy chain constant regions may be modified such that they differ in amino acid sequence from naturally-occurring immunoglobulin molecules.

The term “light chain constant region” includes an amino acid sequence from the antibody light chain. Preferably, the light chain constant region includes at least one of a constant kappa domain and a constant lambda domain.

The term “Fc region” or “Fc fragment” refers to the C-terminal regions of the immunoglobulin heavy chain, which includes at least part of a hinge region, a CH2 domain and a CH3 domain. It mediates the binding of immunoglobulins to host tissues or factors, including the binding of immunoglobulins to Fc receptors located on various cells (for example, effector cells) of the immune system or the binding of immunoglobulins to the first component (C1q) of the classical complement system. The Fc region includes the native sequence Fc region and the variant Fc region.

Generally, the human IgG heavy chain Fc region is a segment from the amino acid residue at the position Cys226 or Pro230 of the human IgG heavy chain Fc region to the carboxy terminus, but its boundaries may vary. The C-terminal lysine of the Fc region (residue 447 according to the EU numbering system) may or may not be present. Fc may also refer to this region in isolation, or in the case of a protein polypeptide including Fc, for example, “binding protein including an Fc region”, and is also referred to as “Fc fusion protein” (for example, antibody or immunoadhesin). The native Fc region of the antibody disclosed in the present disclosure includes mammalian (for example, human) IgG1, IgG2 (IgG2A and IgG2B), IgG3 and IgG4. In some embodiments, there is a single amino acid substitution, insertion and/or deletion of about 10 amino acids per 100 amino acids in the amino acid sequences of two Fc polypeptide chains relative to the sequence of the amino acid sequence of the mammalian Fc polypeptide. In some embodiments, the difference may be changes in Fc that extend the half-life, changes that increase FcRn binding, changes that inhibit Fcy receptor (FcγR) binding and/or changes that decrease or remove ADCC, ADCP and/or CDC.

In IgG, IgA and IgD antibody isotypes, the Fc region contains CH2 and CH3 domains of the antibody's two heavy chains; IgM and IgE Fc regions contain three heavy chain constant domains (CH domains 2-4) in each polypeptide chain.

The term “chimeric antibody” refers to an antibody in which a part of a heavy chain and/or a light chain is identical or homologous to a corresponding sequence derived from a specific species or belonging to a specific antibody type or subtype and the remainder of the chain is identical or homologous to a corresponding sequence derived from another species or belonging to another antibody type or subtype, as well as fragments of such an antibody, as long as they exhibit desired biological activity (U.S. Pat. No. 4,816,567; Morrison S L et al., Proc. Natl. Acad. Sci. U.S.A., 81: 6851-6855). For example, the term “chimeric antibody” may include such an antibody (for example, a human-murine chimeric antibody) in which the heavy and light chain variable regions of the antibody are from a first antibody (for example, a murine antibody) and the heavy and light chain constant regions of the antibody are from a second antibody (for example, a human antibody).

The term “humanized antibody” refers to a genetically engineered non-human antibody whose amino acid sequence has been modified to increase homology to the sequence of the human antibody. Most or all of the amino acids outside the CDR domain of a non-human antibody, for example, a mouse antibody, are substituted by corresponding amino acids from human immunoglobulins, while most or all of the amino acids within one or more CDR regions are not altered. The addition, deletion, insertion, substitution or modification of amino acids is permissible as long as they do not eliminate the ability of the antibody to bind a particular antigen. The “humanized” antibody retains the antigen specificity similar to the antigen specificity of the original antibody. The origin of the CDRs is not particularly limited and may be derived from any animal. For example, CDR regions derived from mouse antibodies, rat antibodies, rabbit antibodies or non-human primate (for example, Cynomolgus monkey) antibodies may be used. The framework region can be obtained by searching the IMGT antibody germline database (http://www.imgt.org/3Dstructure-DB/cgi/DomainGapAlign.cgi) for human antibody germline sequences. Generally, a human germline antibody sequence with high homology to the non-human antibody to be modified is selected as the framework region of the humanized antibody.

The term “hypervariable region”, “CDR” or “complementarity-determining region” refers to amino acid residues of an antibody, which are responsible for antigen binding, and is a discontinuous amino acid sequence. CDR sequences are amino acid residues in the variable region that may be defined by the Kabat, Chothia, IMGT (Lefranc et al., 2003, Dev. Comparat. Immunol., 27:55-77) or AbM (Martin A C R et al., 1989, Proc. Natl. Acad. Sci. U.S.A., 86:9268-9272) method and identified by any CDR sequence determination method well known in the art. For example, the hypervariable region includes the following amino acid residues: amino acid residues from a “complementarity-determining region” or “CDR” defined by sequence comparison, for example, residues at positions 24-34 (LCDR1), 50-56 (LCDR2) and 89-97 (LCDR3) in a light chain variable domain and residues at positions 31-35 (HCDR1), 50-65 (HCDR2) and 95-102 (HCDR3) in a heavy chain variable domain (see Kabat et al., 1991, Sequences of Proteins of Immunological Interest (5th edition), Public Health Service, National Institutes of Health, Bethesda, Md.), and/or amino acid residues from a “hypervariable loop” (HVL) defined according to the structure, for example, residues at positions 26-32(LCDR1), 50-52 (LCDR2) and 91-96 (LCDR3) in the light chain variable domain and residues at positions 26-32 (HCDR1), 53-55 (HCDR2) and 96-101 (HCDR3) in the heavy chain variable domain (see Chothia C and Lesk A M, 1987, J. Mol. Biol., 196:901-917; Chothia C et al., 1989, Nature, 342:878-883). “Framework” residues or “FR” residues refer to variable domain residues other than the hypervariable region residues as defined in the present disclosure. In some embodiments, the antibody or the antigen-binding fragment thereof disclosed in the present disclosure is preferably determined through the Kabat, IMGT or Chothia numbering system. Those skilled in the art may explicitly assign each system to any variable domain sequence without relying on any experimental data beyond the sequence itself. For example, the Kabat residue numbering method of a given antibody may be determined by comparing the sequence of the given antibody to each “standard” numbered sequence. Based on the numbers of the sequences provided herein, the numbering scheme of determining any variable region sequence in the sequence table is entirely within the conventional technical scope of those skilled in the art.

The term “recombinant” refers to a form of a polypeptide or polynucleotide that is not present at the natural state when the polypeptide or the polynucleotide is involved, which, in one non-limiting example, may be implemented by combining polynucleotides or polypeptides that are generally not present together.

The term “vector” refers to a nucleic acid molecule capable of transporting another nucleic acid to which it is linked. One type of vector is a “plasmid” which refers to a circular double-stranded DNA ring to which an additional DNA segment can be linked. Another type of vector is a viral vector in which an additional DNA fragment can be linked to a viral genome. Some vectors can be autonomously replicated in host cells into which these vectors are introduced (for example, a bacterial vector with a bacterial replication starting point and an episomal mammalian vector). Other vectors (for example, non-additional mammalian vectors) may be integrated into the genome of the host cell after introduced into the host cell and replicated together with the genome of the host cell. In addition, some vectors can direct the expression of genes to which they are effectively linked. Such vectors are referred to as “recombinant expression vectors” (or simply referred to as “expression vectors”) herein. Generally, the expression vectors useful in recombinant DNA techniques are typically present in the form of plasmids. However, other forms of expression vectors are also included, such as viral vectors (for example, replication-defective retroviruses, adenoviruses and adeno concomitant viruses), which perform equivalent functions.

The term “isolated antibody molecule” refers to an antibody molecule that has been identified and isolated and/or recovered from components in its natural environment. The contamination components in the natural environment of the antibody molecule are substances that interfere with the diagnostic or therapeutic use of the antibody and may include enzymes, hormones and other protein or non-protein solutes.

The term “isolated” related to nucleic acids (such as DNA or RNA) means that the molecule is isolated from other DNA or RNA which is present as the macromolecule of natural origin. The term “isolated” used herein also means that the nucleic acid or the peptide is substantially free of cellular material, viral material or culture medium when produced by recombinant DNA techniques or substantially free of chemical precursors or other chemicals when chemically synthesized. In addition, the “isolated nucleic acid” refers to a nucleic acid fragment that is not a naturally-occurring fragment and is not found in its natural state. The term “isolated” used herein is also used to refer to cells or polypeptides being isolated from other cellular proteins or tissues. The isolated polypeptide includes purified and recombinant polypeptides.

The term “cross-reaction” refers to the ability of the antibody described herein to bind to antigens from different species. Cross-reactivity may be measured by detecting specific reactivity with purified antigens in binding assays (for example, SPR and ELISA), or detecting the binding to cells physiologically expressing antigens or the interaction with the function of cells physiologically expressing antigens. Examples of assays known in the art for determining the binding affinity include surface plasmon resonance (for example, Biacore) or similar techniques (for example, Kinexa or Octet).

The term “immunobinding” and “immunobinding property” refers to a non-covalent interaction between an immunoglobulin molecule and an antigen (to the antigen, the immunoglobulin is specific). The strength or affinity of the immunobinding interaction may be represented by the equilibrium dissociation constant (K_{D}) of the interaction, where the smaller the K_{D} value, the higher the affinity. The immunobinding property of the selected polypeptide may be quantified using a method known in the art. One method relates to the measurement of rates at which an antigen/antibody complex is formed and dissociated. Both the “association rate constant” (Ka or Kₒₙ) and the “dissociation rate constant” (Kd or K_{off}) may be calculated according to the concentration and actual rates of association and dissociation (see Malmqvist M et al., Nature, 361: 186-187, 1993). The ratio of kd/ka is the dissociation constant K_{D} (see Davies et al., Annual Rev. Biochem., 1990, 59: 439-473). The values of K_{D}, kₐ and k_{d} may be measured using any effective methods. The term “immunogenicity” refers to the ability of a specific substance to elicit an immune response. The term “host cell” refers to a cell in which a vector can proliferate and its DNA can be expressed, where the cell may be a prokaryotic or eukaryotic cell. The term also includes any progeny of the subject host cell. It is to be understood that not all the progeny are identical to the parent cells due to possible mutations occurring in the replication process, and such progeny are included. The host cell includes a prokaryotic cell, a yeast or a mammal cell such as a CHO cell, an NS0 cell or another mammal cell.

The term “identity” refers to the matching of sequences between two polypeptides or between two nucleic acids. When a certain position in each of two sequences for comparison is occupied by the same base group or amino acid monomer subunit (for example, a certain position in each of the two DNA molecules is occupied by adenine, or a certain position in each of the two polypeptides is occupied by lysine), then the molecules are identical at this position. The “percent identity” between two sequences is a function of the number of matching positions of the two sequences divided by the number of positions to be compared and then multiplied by 100. For example, if six of ten positions of the two sequences are matched, the identity between the two sequences is 60%. For example, DNA sequences CTGACT and CAGGTT have a total identity of 50% (three of a total of six positions are matched). Generally, the comparison is made when two sequences are aligned to produce maximum identity. Such alignment may be conveniently implemented through a computer program such as Align program (DNAstar Inc.) or may be implemented using the Needleman and Wunsch methods (Needleman S B and Wunsch C D, 1970, J. Mol. Biol., 48:443-453). The terms “mutated”, “mutant” and “mutation” refer to the substitution, deletion or insertion of one or more nucleotides or amino acids relative to a natural nucleic acid or a polypeptide (that is, used for defining a wild-type reference sequence).

The term “conservative modification” is intended to mean that an amino acid modification does not significantly affect or change the binding characteristics of an antibody including an amino acid sequence. Such conservative modifications include amino acid substitutions, additions and deletions. The modification may be introduced into the antibody disclosed in the present disclosure by using standard techniques known in the art, such as a site-directed mutagenesis and a PCR-mediated mutagenesis. The conservative amino acid substitution refers to the substitution of an amino acid residue with an amino acid residue with a similar side chain. Families of amino acid residues with similar side chains have been described in detail in the art. These families include amino acids with basic side chains (for example, lysine, arginine and histidine), amino acids with acidic side chains (for example, aspartate and glutamine), amino acids with uncharged polar side chains (for example, glycine, asparagine, glutamine, serine, threonine, tyrosine, cysteine and tryptophan), amino acids with non-polar side chains (for example, alanine, valine, leucine, isoleucine, proline, phenylalanine and methionine), amino acids with β-branched side chains (for example, threonine, valine and isoleucine) and amino acids with aromatic side chains (for example, tyrosine, phenylalanine, tryptophan and histidine). Therefore, one or more amino acid residues in the CDR of the antibody disclosed in the present disclosure may be substituted with other amino acid residues from the same side chain family. The antibody disclosed in the present disclosure or a nucleic acid or polynucleotide encoding the antibody disclosed in the present application may be applied to the preparation of a pharmaceutical composition or a sterile composition. For example, the antibody is mixed with a pharmaceutically acceptable carrier, excipient or stabilizer. The pharmaceutical composition may include one antibody or a combination of (for example, two or more different) antibodies disclosed in the present disclosure. For example, the pharmaceutical composition of the present disclosure may include a combination of antibodies or antibody fragments (or immunoconjugates) that have complementary activity and bind to different epitopes on a target antigen. Formulations of therapeutic and diagnostic agents may be prepared by mixing the antibody with the pharmaceutically acceptable carrier, excipient or stabilizer in the form of, for example, lyophilized powder, slurry, an aqueous solution or a suspension. The term “pharmaceutically acceptable” means that a molecule, molecule fragment or composition does not produce unfavorable, allergic or other adverse effects when properly administered to animals or humans. Specific examples of some substances that may act as the pharmaceutically acceptable carriers or components thereof include sugars (for example, lactose), starch, cellulose and its derivatives, vegetable oils, gelatin, polyols (for example, propylene glycol), alginic acid and the like. The antibody of the present disclosure or the nucleic acid or polynucleotide encoding the antibody of the present application may be used alone or in combination with one or more other therapeutic agents, and the therapeutic agents may be, for example, vaccines. The term “pharmaceutically acceptable carrier and/or excipient and/or stabilizer” refers to a carrier and/or excipient and/or stabilizer which is pharmacologically and/or physiologically compatible with the subject and the active ingredient and which is non-toxic to the cell or mammal exposed to such a carrier and/or excipient and/or stabilizer at the dosage and concentration employed. Examples include, but are not limited to, pH regulators, surfactants, adjuvants, ionic strength enhancers, diluents, reagents to maintain osmotic pressure, reagents to delay absorption, and preservatives. For example, pH adjusting agents include, but are not limited to, phosphate buffers. Surfactants include, but are not limited to, cationic surfactants, anionic surfactants or nonionic surfactants, for example, Tween-80. Ionic strength enhancers include, but are not limited to, sodium chloride. Preservatives include, but are not limited to, various antibacterial reagents and antifungal reagents, such as parabens, chlorobutanol, phenol and sorbic acid. Reagents to maintain osmotic pressure include, but are not limited to, sugars, NaCl and analogs thereof. Reagents to delay absorption include, but are not limited to, monostearate and gelatin. Diluents include, but are not limited to, water, aqueous buffers (for example, buffered saline), alcohols, and polyols (for example, glycerol). Preservatives include, but are not limited to, various antibacterial reagents and antifungal reagents, such as thiomersal, 2-phenoxyethanol, parabens, chlorobutanol, phenol and sorbic acid. Stabilizers have the meaning commonly understood by those skilled in the art and are those capable of stabilizing the desired activity of the active ingredient in a drug, including, but not limited to, sodium glutamate, gelatin, SPGA, sugars (for example, sorbitol, mannitol, starch, sucrose, lactose, dextran or glucose), amino acids (for example, glutamic acid or glycine), proteins (for example, dry whey, albumin or casein), or degradation products thereof (for example, lactalbumin hydrolysate). The term “effective amount” used herein refers to an amount sufficient to obtain or at least partially obtain the desired effect. For example, a prophylactically (for example, tumor, infection or autoimmune disease) effective amount refers to an amount sufficient to prevent, arrest or delay the onset of a disease (for example, tumor, infection or autoimmune disease); a therapeutically effective amount refers to an amount sufficient to cure or at least partially arrest the disease and complications thereof in a patient already suffering from the disease. It is well within the ability of those skilled in the art to determine such effective amounts. For example, the amount effective for therapeutic use depends on the severity of the disease to be treated, the overall state of the patient's own immune system, the general condition of the patient such as age, weight and sex, the mode of administration of the drug, and other treatments administered concurrently. The term “immune cells” used herein include cells of hematopoietic origin that function in immune responses, for example, lymphocytes such as B cells and T cells, natural killer cells, and myeloid cells such as monocytes, macrophages, eosinophils, mast cells, basophils and granulocytes. The term “immune response” used herein refers to an action of immune cells (such as lymphocytes, antigen-presenting cells, phagocytes or granulocytes) and soluble macromolecules (including antibodies, cytokines and complements) produced by immune cells or the liver. Such action results in selective injuries and damages to invasive pathogens, cells or tissues infected with pathogens, cancer cells, or normal human cells or tissues in the context of autoimmunity or pathological inflammation, or their clear from human bodies. In the present disclosure, the term “antigen-specific T cell response” refers to an immune response produced by the T cell, which occurs when the T cell-specific antigen stimulates the T cell. Non-limiting examples of responses produced by the T cell upon antigen-specific stimulation include the proliferation of T cells and the production of cytokines such as IL-2.

The term “effector function” refers to biological activities that can be attributed to the biological activities of the antibody Fc region (a native sequence Fc region or an amino acid sequence variant Fc region) and that vary with antibody isotypes. Examples of antibody effector functions include, but are not limited to, Fc receptor binding affinity, antibody-dependent cell-mediated cytotoxicity (ADCC), complement-dependent cytotoxicity (CDC), antibody-dependent cell-mediated phagocytosis (ADCP), downregulation of cell surface receptors (for example, B cell receptors), B cell activation, cytokine secretion and half-life/clearance rate of antibodies and antigen-antibody complexes. Methods of altering the effector function of antibodies are known in the art, for example, the effector function of antibodies may be altered by introducing mutations in the Fc region.

The term “antibody-dependent cell-mediated cytotoxicity (ADCC)” refers to a cytotoxic form in which Ig binds to Fc receptors (FcRs) on cytotoxic cells (for example, natural killer (NK) cells, neutrophils or macrophages) to enable these cytotoxic effector cells to specifically bind to antigen-attached target cells and then secret cytotoxins to kill the target cells. Methods for detecting the ADCC activity of an antibody are known in the art, for example, the ADCC activity may be detected by measuring the binding activity between an antibody to be tested and an Fc receptor (for example, CD16a).

The term “complement-dependent cytotoxicity (CDC)” refers to a cytotoxic form that activates the complement cascade by binding the complement component C1q to the antibody Fc. Methods for detecting the CDC activity of an antibody are known in the art, for example, the CDC activity may be detected by measuring the binding activity between an antibody to be tested and an Fc receptor (for example, C1q).

Embodiments of the present disclosure are further illustrated through the following examples, but it is to be appreciated by those skilled in the art that the following figures and examples are merely illustrative of the present disclosure and are not intended to further limit the present disclosure.

### BRIEF DESCRIPTION OF DRAWINGS

FIG. 1 shows the determination of the binding ability of anti-B7-H3 murine antibodies to human B7-H3 antigens.

FIG. 2 shows the determination of the cross-reactivity of anti-B7-H3 murine antibodies with mouse B7-H3 antigens.

FIG. 3 shows the determination of the cross-reactivity of anti-B7-H3 murine antibodies with monkey B7-H3 antigens.

### DETAILED DESCRIPTION

Embodiments of the present disclosure are described with reference to the following examples that are meant to be illustrative (not to be limiting) to describe the present disclosure.

Unless otherwise specified, the molecular biology experimental methods and immunoassays adopted in the present disclosure are performed essentially referring to those described in J. Sambrook et al., Molecular Cloning: A Laboratory Manual, 2nd edition, Cold Spring Harbor Laboratory, 1989, and F. M. Ausubel et al., Short Protocols in Molecular Biology, 3rd edition, John Wiley&Sons, Inc. 1995; restriction endonucleases are used in accordance with the conditions recommended by the product manufacturer. It is to be appreciated by those skilled in the art that the embodiments described herein are provided by way of example and should not be intended to limit the scope of the present disclosure to these embodiments.

### Example 1 Preparation of Murine Monoclonal Antibodies Against Human B7-H3

Human B7-H3 antigen (protein sequence: Uniprot entry No. Q5ZPR3) was fully emulsified with a Freund's complete adjuvant and then was immunized to male Balb/C mice, 50 μg/mouse, by multisite immunization in an immunization cycle of once three weeks. On the tenth day after the third immunization, blood was collected from the caudal vein, the titer of the antibody against human B7-H3 in plasma was tested by ELISA to monitor the degree of immune response in mice, and then a mouse with the highest titer of the antibody against human B7-H3 was subjected to booster immunization once three days before fusion. Three days later, the mouse was sacrificed, and its spleen was removed from the mouse and fused with a mouse Sp2/0 myeloma cell strain. 2×10⁸ Sp2/0 cells were fused with 2×10⁸ spleen cells in a solution of 50% polyethylene glycol (with a molecular weight of 1450) and 5% dimethyl sulfoxide (DMSO). The number of spleen cells was adjusted to 5×10⁵/mL by using an Iscove's medium (containing 10% fetal bovine serum, 100 U/mL penicillin, 100 μg/mL streptomycin, 0.1 mM hypoxanthine, 0.4 μM aminopterin and 16 μg thymidine), and 0.3 mL was added to the wells of a 96-well plate and placed in an incubator at 5% CO₂ and 37° C. After ten days of culture, antibody clones that bound to B7-H3 with high affinity in the supernatant were detected separately by high-throughput ELISA. The fused cells in the wells of the above monoclonal antibodies were subcloned and further screened to obtain hybridoma cell strains #152 and #272.

Clones that produced a specific antibody were cultured in an RPMI 1640 medium supplemented with 10% FCS. When the cell density reached approximately 5×10⁵ cells/mL, the medium was replaced with a serum-free medium. After two to four days, the cultured medium was centrifuged to collect the culture supernatant. The antibody was purified with protein G column. The eluent of the monoclonal antibody was dialyzed against 150 mM NaCl. The dialyzed solution was filtered and sterilized through a 0.2 μm filter to obtain the purified murine monoclonal antibodies mAb152 and mAb272 to be tested.

### Example 2 Determination of Abilities of Murine Antibodies to Bind to a Human B7-H3 Antigen by ELISA

Plates were coated with 100 μL of 0.1 μg/mL human B7-H3 (purchased from Acro Biosystems) at room temperature overnight. The coating solution was discarded, and the wells were blocked for 0.5 hours with skimmed milk dissolved in phosphate buffered saline (PBS) and washed with PBS containing 0.05% Tween-20 (PBST). The murine antibodies mAb152 and mAb272 against human B7-H3 to be tested were then diluted to appropriate concentrations, added to plates, 50 μL per well, and incubated at room temperature for 1 hour. The wells were washed with 0.05% PBST, and then 50 μL of HRP-labeled goat anti-mouse IgG polyclonal antibodies (purchased from Jackson Laboratory) was added to each well as the detection antibody and incubated at 37° C. for 1 hour. The cells were washed three times with 0.05% PBST, TMB was added to the plates, 100 μL/well, and color development was performed at room temperature for 5 min. 0.2 M H₂SO₄ was added, 50 μL/well, to stop the color development reaction. The light absorbance values at 450 nm/620 nm were measured by a microplate reader. A graph was drawn by software GraphPad Prism 7 with the absorbance as the Y-axis and the antibody concentration as the X-axis.

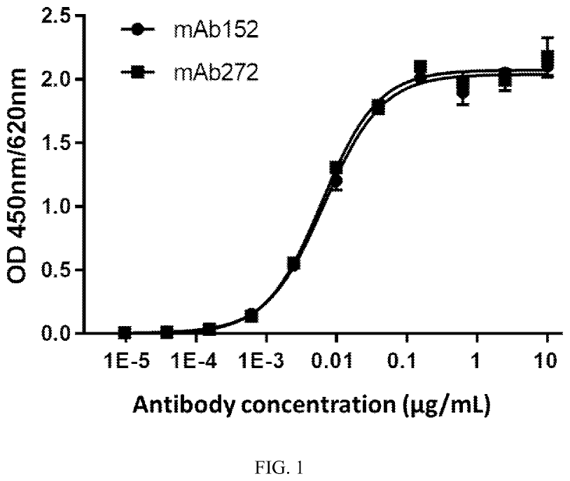

The results are shown in FIG. 1, both murine antibodies mAb152 and mAb272 had high affinity with human B7-H3, and the EC₅₀ values for the binding of mAb152 and mAb272 to human B7-H3 molecules were 6.69 ng/ml and 6.16 ng/ml, respectively.

### Example 3 Affinity Determination and Kinetic Study of B7-H3 Murine Antibodies

The binding affinity constants of the purified murine monoclonal antibodies to the antigen B7-H3 were determined by bio-layer interferometry (BLI) with a ForteBio Octet RED&QK system from PALL Corporation. Concentration gradients of a multichannel parallel quantitative analysis were set to: 3.125, 6.25, 12.5, 25, 50 and 100 nM, and His-labeled human B7-H3 (10 μg/mL) was coupled to a Ni-NTA sensor. The affinity determination results are shown in Table 1 and show that the murine monoclonal antibodies had very high binding affinity to human B7-H3, which can be of the order of 10⁻¹¹ M.

TABLE 1
Affinity determination results of murine monoclonal antibodies

| Antibody | K_{D} (M) | ka (1/Ms) | kd (1/s) |
|---|---|---|---|
| mAb152 | 1.496E−11 | 2.067E+05 | 3.092E−06 |
| mAb272 | <1.0E−12 | 2.201E+05 | <1.0E−07 |

### Example 4 Evaluation of In Vitro Biological Functions of Anti-B7-H3 Monoclonal Antibodies

### 4.1 Determination of the Binding Activity of Anti-B7-H3 Murine Antibodies to Target Cells

Binding activities of the murine antibodies mAb152 and mAb272 to breast cancer cells Hs578T, MDA-MB-436, MDA-MB-468, MDA-MB-231 and MDA-MB-453 (purchase from Nanjing Cobioer Biotechnology Co., Ltd.) and human colon cancer cell HT-29 (purchase from the cell bank of Chinese Academy of Sciences, Shanghai) were detected by flow cytometry.

The tumor cells Hs578T, MDA-MB-436, MDA-MB-468, MDA-MB-231, MDA-MB-453 and HT-29 were cultured, then digested with 0.25% trypsin, and centrifuged to collect cells. The collected cells were resuspended with 1% PBSB to adjust the cell density to 2×10⁶ cells/ml, placed in 96-well plates 100 μL (2×10⁵ cells) per well, and blocked for 0.5 hours at 4° C. The blocked cells were centrifuged to discard the supernatant, and a series of diluted antibodies mAb152 and mAb272 were added to the cells. The cells were incubated for 1 hour at 4° C., then centrifuged to discard the supernatant, and washed three times with a PBS solution with 1% BSA (PBSB). Diluted AF64-labeled goat anti-human IgG antibodies (Jackson, Cat. No. 109-605-088) were added to the cells, and the cells were incubated for 1 hour at 4° C. in the dark. The obtained cells were centrifuged to discard the supernatant and then washed twice with 1% PBSB, and cells in each well were resuspended with 100 μL of 1% paraformaldehyde (PF). The signal intensity was detected by flow cytometry. The analysis was performed with the mean fluorescence intensity (MFI) as the Y-axis and the antibody concentration as the X-axis through the software GraphPad Prism 7 to calculate the Kd values for the binding of the murine monoclonal antibodies to the tumor cells.

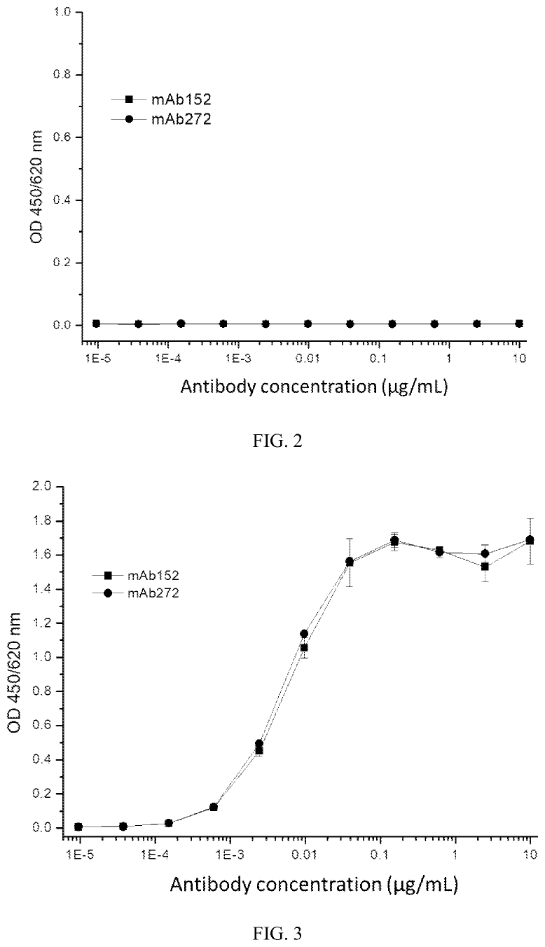

The results show that the murine monoclonal antibodies mAb152 and mAb272 had strong binding activity to Hs578T, MDA-MB-436 and MDA-MB-468 cells and had weak binding activity to MDA-MB-231, MDA-MB-453 and HT-29 cells. FIG. 2-1 shows binding curves of the antibodies to the tumor cells. As shown in Tables 2 and 3, the Kd for the binding of the monoclonal antibodies mAb152 and mAb272 to the tumor cells was between 0.2-5.2 nM.

TABLE 2
Binding constants of the monoclonal
antibody mAb152 to tumor cells

| | | MDA- | MDA- | MDA- | MDA- | |
|---|---|---|---|---|---|---|
| | Hs578T | MB-436 | MB-468 | MB-231 | MB-453 | HT-29 |
| Kd (nM) | 0.8346 | 5.046 | 2.597 | 0.2265 | 0.2729 | 1.255 |

TABLE 3
Binding constants of the monoclonal
antibody mAb272 to tumor cells

| | | MDA- | MDA- | MDA- | MDA- | |
|---|---|---|---|---|---|---|
| | Hs578T | MB-436 | MB-468 | MB-231 | MB-453 | HT-29 |
| Kd (nM) | 1.196 | 5.110 | 3.244 | 0.2044 | 0.2388 | 2.350 |

### 4. 2 Cross-Reactivity of Anti-B7-H3 Murine Monoclonal Antibodies

The cross-reactivity of the anti-B7-H3 murine monoclonal antibodies with mouse and Cynomolgus monkey B7-H3 antigens was detected in this example.

Specific experimental procedures are as follows: The plates were coated with 100 μL of 0.1 μg/mL Cynomolgus monkey B7-H3 and murine B7-H3 proteins (Sino Biological Inc. Beijing) at room temperature overnight. The coating solution was discarded, and the wells were blocked for 0.5 hours with skimmed milk dissolved in phosphate buffered saline (PBS) and washed with PBS containing 0.05% Tween-20. 50 μL of purified HRP-labeled B7-H3 monoclonal antibodies was added to each well and incubated at room temperature for 1 hour. The cells were washed five times with PBS containing 0.05% Tween-20, 100 μL of TMB was added to each well, and color development was performed at room temperature for 5 min. 50 μL of 2N H₂SO₄ was added to each well to stop the color development reaction. The values at 450 nm were measured by a microplate reader. The results were imported into Graph Prism to calculate EC₅₀ values.

As shown in FIGS. 2 and 3, the two B7-H3 monoclonal antibodies did not bind to the murine B7-H3 antigen, but both bound to the monkey B7-H3 antigen.

### Example 5 Humanization of Murine Antibodies Against Human B7-H3

The murine antibodies were humanized by CDR grafting. The basic principle of CDR grafting is: grafting the CDR regions of a murine antibody onto a human antibody template, and introducing the stable CDR conformation and several or some key murine antibody FR region residues of interest for the antigenic-antibody binding onto the human antibody template (backmutations), thereby reducing the immunogenicity of the murine antibody and maintaining the affinity of the murine antibody. In addition to the above-mentioned CDR grafting procedure, the isoelectric point (PI), hydrophobic aggregation, post-translational modification (PTM, such as glycosylation, cleavage, isomerization sites, etc.) and immunogenicity of the CDR-grafted humanized antibody were further be calculated, and the amino acids causing problems in these four aspects were mutated to enable the humanized antibody to fully exert the pharmacological effect in clinical use.

The specific procedure for antibody humanization is as follows. The IMGT human antibody germline database (http://www.imgt.org/3Dstructure-DB/cgi/DomainGapAlign.cgi) was searched to obtain a human antibody template with high homology to the murine antibody. The CDR regions were annotated by Discovery Studio for the murine antibody and the human antibody template, and the CDR regions were defined according to the Kabat or IMGT scheme. The six CDR regions of the human antibody template were replaced with six CDR regions of the murine antibody, respectively. Each CDR region in each of the six grafted CDR regions may be an amino acid region defined by Kabat or an amino acid region defined by IMGT. Backmutations of the FR regions from the murine antibody to the humanized template were performed after CDR grafting. The stable antibody CDR conformation and the several or some key murine antibody FR region amino acids of interest for the antigenic-antibody binding include four types of amino acid residues: 1) amino acids embedded in the CDR region 6 Å below the surface of the antibody; 2) amino acids exposed to the surface of the antibody in the CDR region within 6 Å; 3) interfacial amino acids between the antibody light and heavy chain domains; and 4) vernier zone residues (Foote J and Winter G, 1992, J. Mol. Biol., 224:487-499) of the stable antibody CDR conformation. The above-mentioned four key murine antibody FR region residues were determined by constructing a murine antibody three-dimensional structural model. For these four types of amino acids of the human template that were not identical to the murine antibody sequence, amino acids of interest for maintaining the CDR conformation and antigen-antibody binding were selected by three-dimensional structure analysis for amino acid grafting or substitution from the murine antibody to the human template. Then, the humanized antibody produced after the grifting of the four types of amino acids was further calculated for isoelectric point, hydrophobic aggregation, post-translational modification and immunogenicity, and the problem amino acids were mutated, thereby obtaining the final humanized antibody sequence.

TABLE 4
CDR sequences for exemplary anti-B7-H3 murine and humanized antibodies

| Antibody No. | CDR | Kabat | IMGT |
|---|---|---|---|
| mAb152/mAb272 | HCDR1 | AYWMN | GYTFTAYW |
| | | (SEQ ID NO: 5) | (SEQ ID NO: 11) |
| | HCDR2 | RIDPYDSETRYNQNFRD | IDPYDSET |
| | | (SEQ ID NO: 6) | (SEQ ID NO: 12) |
| | HCDR3 | GVRIFDY | ARGVRIFDY |
| | | (SEQ ID NO: 7) | (SEQ ID NO: 13) |
| | LCDR1 | RTSENIDYTLA | ENIDYT |
| | | (SEQ ID NO: 8) | (SEQ ID NO: 14) |
| | LCDR2 | NANTLED | NAN |
| | | (SEQ ID NO: 9) | (SEQ ID NO: 15) |
| | LCDR3 | KQAYDVPRT | KQAYDVPRT |
| | | (SEQ ID NO: 10) | (SEQ ID NO: 10) |
| AB125/AB126 | HCDR1 | AYWMN | GYTFTAYW |
| | | (SEQ ID NO: 5) | (SEQ ID NO: 11) |
| | HCDR2 | RIEPYDSETRYNQNFRD | IEPYDSET |
| | | (SEQ ID NO: 24) | (SEQ ID NO: 26) |
| | HCDR3 | GVRIFDY | ARGVRIFDY |
| | | (SEQ ID NO: 7) | (SEQ ID NO: 13) |
| | LCDR1 | RTSENIDYTLA | ENIDYT |
| | | (SEQ ID NO: 8) | (SEQ ID NO: 14) |
| | LCDR2 | NANTLEE | NAN |
| | | (SEQ ID NO: 25) | (SEQ ID NO: 15) |
| | LCDR3 | KQAYDVPRT | KQAYDVPRT |
| | | (SEQ ID NO: 10) | (SEQ ID NO: 10) |
| AB127/AB128 | HCDR1 | AYWMN | GYTFTAYW |
| | | (SEQ ID NO: 5) | (SEQ ID NO: 11) |
| | HCDR2 | RIDPYDSETRYNQNFRD | IDPYDSET |
| | | (SEQ ID NO: 6) | (SEQ ID NO: 12) |
| | HCDR3 | GVRIFDY | ARGVRIFDY |
| | | (SEQ ID NO: 7) | (SEQ ID NO: 13) |
| | LCDR1 | RTSENIDYTLA | ENIDYT |
| | | (SEQ ID NO: 8) | (SEQ ID NO: 14) |
| | LCDR2 | NANTLEE | NAN |
| | | (SEQ ID NO: 25) | (SEQ ID NO: 15) |
| | LCDR3 | KQAYDVPRT | KQAYDVPRT |
| | | (SEQ ID NO: 10) | (SEQ ID NO: 10) |

According to the above-mentioned method, four humanized antibodies, designated as AB125, AB126, AB127 and AB128, were constructed based on the CDRs of the murine antibodies mAb152 and mAb272, respectively. The CDR regions and the heavy and light chain variable region amino acid sequences included in the variable regions of the murine antibody and the humanized antibody are shown in Table 4 and Table 5.

TABLE 5
Amino acid sequences of the variable regions
of murine and humanized antibodies

| | VH amino acid sequence | VL amino acid sequence |
|---|---|---|
| mAb152 | SEQ ID NO: 1 | SEQ ID NO: 2 |
| mAb272 | SEQ ID NO: 3 | SEQ ID NO: 4 |
| AB125 | SEQ ID NO: 16 | SEQ ID NO: 17 |
| AB126 | SEQ ID NO: 18 | SEQ ID NO: 19 |
| AB127 | SEQ ID NO: 20 | SEQ ID NO: 21 |
| AB128 | SEQ ID NO: 22 | SEQ ID NO: 23 |

To obtain a full-length antibody sequence consisting of two heavy chains and two light chains, the VH and VL sequences shown in Table 5 were spliced or assembled with the sequences of a heavy chain constant region (preferably, from human IgG1, IgG2 or IgG4) and a light chain constant region (preferably, from a human κ light chain) using conventional techniques. Preferably, the heavy chain constant region was a human wild-type heavy chain constant region or a mutant thereof.

### Example 6 Construction, Expression and Preparation of Anti-Human B7-H3 Antibodies

The encoding cDNA according to the heavy and light chain sequences obtained in the above examples was designed and inserted into a pCMAB2M eukaryotic expression vector to construct a humanized expression vector. The expression vector plasmid contained the cytomegalovirus early gene promoter-enhancer required for the high-level expression in mammalian cells. The vector plasmid also contained a selective marker so that kanamycin resistance may be present in bacteria and G418 resistance may be present in mammalian cells. In addition, the vector plasmid also contained dihydrofolate reductase (DHFR) genes so that antibody genes and the DHFR genes could be co-amplified in the presence of methotrexate (MTX) in appropriate host cells.

The recombinant expression vector plasmids constructed above were transfected into a mammalian host cell line to express the humanized antibody. To maintain stable and high-level expression, the preferred host cell line was a DHFR-deficient Chinese hamster ovary (CHO) cell (see U.S. Pat. No. 4,818,679). A preferred transfection method was electroporation. Other methods, including calcium phosphate co-precipitation and lipofection may also be used. During electroporation, 2×10⁷ cells were added to a cuvette with a GenePulser Electroporator (Bio-Rad Laboratories) with an electric field of 300 V and capacitance of 1050 μFd and suspended in 0.8 mL of PBS, and 20 μg of expression vector plasmids were also added. After two-day transfection, 0.2 mg/mL G418 and 200 nM MTX (Sigma) were added. To achieve the high-level expression of fusion proteins, DHFR genes inhibited by MTX were used for co-amplification of the transfected antibody genes. Transfectants were subcloned by the limiting dilution method, and the secretion rate of each cell line was determined by ELISA. Cell strains expressing antibodies at high levels were screened. The conditioned medium of antibodies was collected for the determination of in vitro and in vivo antibody biological activity.

Though the preferred examples of the present disclosure have been illustrated and described, it is to be understood that those skilled in the art can make various changes in accordance with the teachings herein without departing from the scope of the present disclosure.

All the publications mentioned in the present disclosure are incorporated herein by reference as if each publication is separately incorporated herein by reference. In addition, it should be understood that those skilled in the art, who have read the preceding content of the present disclosure, can make various changes or modifications on the present disclosure, and these equivalent forms fall within the scope of the appended claims of the present application.

1. An antibody or an antigen-binding fragment thereof specifically binding to B7-H3, wherein the antibody or the antigen-binding fragment thereof comprises a heavy chain variable region (VH) comprising at least one, two or three complementarity-determining regions (CDRs) selected from the group consisting of:
(i) HCDR1 having a sequence as shown in SEQ ID NO: 5 or 11 or having a sequence with one or more amino acid substitutions, deletions or additions (for example, one, two or three substitutions, deletions or additions) relative to any of the above sequences;
(ii) HCDR2 having a sequence as shown in SEQ ID NO: 6, 12, 24 or 26 or having a sequence with one or more amino acid substitutions, deletions or additions (for example, one, two or three substitutions, deletions or additions) relative to any of the above sequences; and
(iii) HCDR3 having a sequence as shown in SEQ ID NO: 7 or 13 or having a sequence with one or more amino acid substitutions, deletions or additions (for example, one, two or three substitutions, deletions or additions) relative to any of the above sequences; and/or
the antibody or the antigen-binding fragment thereof comprises a light chain variable region (VL) comprising at least one, two or three CDRs selected from the group consisting of:
(iv) LCDR1 having a sequence as shown in SEQ ID NO: 8 or 14 or having a sequence with one or more amino acid substitutions, deletions or additions (for example, one, two or three substitutions, deletions or additions) relative to any of the above sequences;
(v) LCDR2 having a sequence as shown in SEQ ID NO: 9, 15 or 25 or having a sequence with one or more amino acid substitutions, deletions or additions (for example, one, two or three substitutions, deletions or additions) relative to any of the above sequences; and
(vi) LCDR3 having a sequence as shown in SEQ ID NO: 10 or having a sequence with one or more amino acid substitutions, deletions or additions (for example, one, two or three substitutions, deletions or additions) relative to the above sequence;
preferably, the substitutions in any one of (i) to (vi) are conservative substitutions.

2. The antibody or the antigen-binding fragment thereof according to claim 1, wherein the antibody or the antigen-binding fragment thereof comprises three VH variable region CDRs and three VL variable region CDRs selected from the group consisting of:
(i) HCDR1, HCDR2, HCDR3, LCDR1, LCDR2, and LCDR3 each having a sequence as shown in SEQ ID NO: 5, 6, 7, 8, 9 or 10 or having a sequence with one or more amino acid substitutions, deletions or additions (for example, one, two or three substitutions, deletions or additions) relative to any of the above sequences;
(ii) HCDR1, HCDR2, HCDR3, LCDR1, LCDR2, and LCDR3 each having a sequence as shown in SEQ ID NO: 5, 24, 7, 8, 25 or 10 or having a sequence with one or more amino acid substitutions, deletions or additions (for example, one, two or three substitutions, deletions or additions) relative to any of the above sequences;
(iii) HCDR1, HCDR2, HCDR3, LCDR1, LCDR2, and LCDR3 each having a sequence as shown in SEQ ID NO: 5, 6, 7, 8, 25 or 10 or having a sequence with one or more amino acid substitutions, deletions or additions (for example, one, two or three substitutions, deletions or additions) relative to any of the above sequences;
(iv) HCDR1, HCDR2, HCDR3, LCDR1, LCDR2, and LCDR3 each having a sequence as shown in SEQ ID NO: 11, 12, 13, 14, 15 or 10 or having a sequence with one or more amino acid substitutions, deletions or additions (for example, one, two or three substitutions, deletions or additions) relative to any of the above sequences; and
(v) HCDR1, HCDR2, HCDR3, LCDR1, LCDR2, and LCDR3 each having a sequence as shown in SEQ ID NO: 11, 26, 13, 14, 15 or 10 or having a sequence with one or more amino acid substitutions, deletions or additions (for example, one, two or three substitutions, deletions or additions) relative to any of the above sequences.

3. The antibody or the antigen-binding fragment thereof according to claim 2, wherein the antibody or the antigen-binding fragment thereof is murine or chimeric, the heavy chain variable region of the antibody or the antigen-binding fragment thereof comprises a heavy chain FR region of murine IgG1, IgG2, IgG3 or a variant thereof, and the light chain variable region of the antibody or the antigen-binding fragment thereof comprises a light chain FR region of a murine κ chain, a murine λ chain or a variant thereof, preferably, the antibody or the antigen-binding fragment thereof comprises VH and VL sequences selected from the group consisting of:
(i) a VH domain comprising an amino acid sequence as shown in SEQ ID NO: 1 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain comprising an amino acid sequence as shown in SEQ ID NO: 2 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and
(ii) a VH domain comprising an amino acid sequence as shown in SEQ ID NO: 3 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain comprising an amino acid sequence as shown in SEQ ID NO: 4 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence.

4. The antibody or the antigen-binding fragment thereof according to claim 2, wherein the antibody or the antigen-binding fragment thereof is humanized, and preferably, the antibody or the antigen-binding fragment thereof comprises VH and VL sequences selected from the group consisting of:
(i) a VH domain comprising an amino acid sequence as shown in SEQ ID NO: 16 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain comprising an amino acid sequence as shown in SEQ ID NO: 17 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence;
(ii) a VH domain comprising an amino acid sequence as shown in SEQ ID NO: 18 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain comprising an amino acid sequence as shown in SEQ ID NO: 19 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence;
(iii) a VH domain comprising an amino acid sequence as shown in SEQ ID NO: 20 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain comprising an amino acid sequence as shown in SEQ ID NO: 21 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and
(iv) a VH domain comprising an amino acid sequence as shown in SEQ ID NO: 22 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence; and a VL domain comprising an amino acid sequence as shown in SEQ ID NO: 23 or having a sequence that is substantially identical to (for example, is at least 80%, 85%, 90%, 92%, 95%, 97%, 98%, 99% or more similar to or has one or more amino acid substitutions (for example, conservative substitutions) than) the above sequence.

5. The antibody or the antigen-binding fragment thereof according to claim 4, wherein the antibody further comprises a heavy chain constant region and a light chain constant region derived from a human immunoglobulin; preferably, the heavy chain constant region is selected from heavy chain constant regions of human IgG1, IgG2, IgG3, and IgG4, and the heavy chain constant region has a natural sequence or a sequence having one or more amino acid substitutions, deletions or additions relative to a natural sequence from which the heavy chain constant region is derived; the light chain constant region is preferably a constant region of a human κappa chain.

6. A DNA molecule encoding the antibody or the antigen-binding fragment thereof according to claim 1.

7. A pharmaceutical composition, comprising the antibody or the antigen-binding fragment thereof according to claim 1, and a pharmaceutically acceptable excipient, carrier or diluent.

8. A method for preparing the antibody or the antigen-binding fragment thereof according to claim 1, comprising: (a) obtaining a gene of the antibody or the antigen-binding fragment thereof to construct an expression vector of the antibody or the antigen-binding fragment thereof, (b) transfecting the expression vector into a host cell by a genetic engineering method; (c) culturing the host cell under conditions that allow the antibody or the antigen-binding fragment thereof to be generated; and (d) separating and purifying the generated antibody or the antigen-binding fragment thereof;
wherein the expression vector in step (a) is one or more selected from plasmids, bacteria, and viruses;
wherein the host cell into which the constructed vector is transfected by the genetic engineering method in step (b) comprises a prokaryotic cell, a yeast or a mammalian cell, such as a CHO cell, an NS0 cell or another mammalian cell; and
the antibody or the antigen-binding fragment thereof is separated and purified in step (d) by a conventional immunoglobulin purification method comprising protein A affinity chromatography and ion exchange, hydrophobic interaction chromatography or molecular sieve.

9. (canceled)

10. A bispecific molecule comprising the antibody or the antigen-binding fragment thereof according to claim 1.

11. A method for prevention and/or treatment of a tumor, comprising administering an effective amount of the antigen-binding fragment thereof according to claim 1 to subject in need thereof;
preferably, the tumor is selected from a solid tumor or a hematological tumor; wherein the tumor comprise, but are not limited to, lung cancer (for example, lung adenocarcinoma or non-small cell lung cancer (NSCLC)), melanoma (for example, advanced melanoma), renal cancer (for example, renal cell carcinoma), liver cancer (for example, hepatocellular carcinoma), myeloma (for example, multiple myeloma), osteosarcoma, prostate cancer, bladder cancer, urethral cancer, breast cancer, ovarian carcinoma, colorectal cancer, pancreatic cancer, head and neck cancer (for example, head and neck squamous-cell carcinoma (HNSCC)), gastric-esophageal cancer (for example, esophageal squamous-cell carcinoma), mesothelioma, nasopharyngeal cancer, thyroid cancer, cervical cancer, neuroblastoma, glioma, diffuse large B-cell lymphoma, T-cell lymphoma, B-cell lymphoma, non-Hodgkin's lymphoma, myeloid leukemia, chronic lymphocytic leukemia, and acute lymphocytic leukemia.


SEQUENCE LISTING (26 sequences)

SEQ ID NO: 1 - protein, 116 aa, Artificial sequence
  Amino acid sequence of heavy chain variable region of mAb152
QVQLQQPGAELVRPGASVKLSCKASGYTFTAYWMNWVKQRPDQGLEWIGRIDPYDSETRYNQNFRDKARLTVDKSSSTAYMQLSSLTSEDSAVFYCARGVRIFDYWGQGTTLTVSS

SEQ ID NO: 2 - protein, 107 aa, Artificial sequence
  Amino acid sequence of light chain variable region of mAb152
DIQMTQSPASLAASVGETVTITCRTSENIDYTLAWYQQKPGKSPQLLIYNANTLEDGVPSRFSGSGSGTHFSLKINSMQPEDTATYFCKQAYDVPRTFGGGTKLEIK

SEQ ID NO: 3 - protein, 116 aa, Artificial sequence; identical to SEQ ID NO: 1
  Amino acid sequence of heavy chain variable region of mAb272

SEQ ID NO: 4 - protein, 107 aa, Artificial sequence
  Amino acid sequence of light chain variable region of mAb272
DIQMTQSPASLAASVGETVTITCRTSENIDYTLAWYQQKPGKSPQLLIYNANTLEDGVPSRFSGSGSGTLFSLKINNMQPEDTATYFCKQAYDVPRTFGGGTKLEIK

SEQ ID NO: 5 - protein, 5 aa, Artificial sequence
  HCDR1
AYWMN

SEQ ID NO: 6 - protein, 17 aa, Artificial sequence
  HCDR2
RIDPYDSETRYNQNFRD

SEQ ID NO: 7 - protein, 7 aa, Artificial sequence
  HCDR3
GVRIFDY

SEQ ID NO: 8 - protein, 11 aa, Artificial sequence
  LCDR1
RTSENIDYTLA

SEQ ID NO: 9 - protein, 7 aa, Artificial sequence
  LCDR2
NANTLED

SEQ ID NO: 10 - protein, 9 aa, Artificial sequence
  LCDR3
KQAYDVPRT

SEQ ID NO: 11 - protein, 8 aa, Artificial sequence
  HCDR1
GYTFTAYW

SEQ ID NO: 12 - protein, 8 aa, Artificial sequence
  HCDR2
IDPYDSET

SEQ ID NO: 13 - protein, 9 aa, Artificial sequence
  HCDR3
ARGVRIFDY

SEQ ID NO: 14 - protein, 6 aa, Artificial sequence
  LCDR1
ENIDYT

SEQ ID NO: 15 - protein, 3 aa, Artificial sequence
  LCDR2
NAN

SEQ ID NO: 16 - protein, 116 aa, Artificial sequence
  Amino acid sequence of heavy chain variable region of AB125
EVQLVQSGAEVKKPGSSVKVSCKASGYTFTAYWMNWVRQAPGQGLEWIGRIEPYDSETRYNQNFRDRATLTVDKSTSTAYMELSSLRSEDTAVYYCARGVRIFDYWGQGTTVTVSS

SEQ ID NO: 17 - protein, 107 aa, Artificial sequence
  Amino acid sequence of light chain variable region of AB125
DIQMTQSPSSLSASVGDRVTIRCRTSENIDYTLAWYQQKPGKSPKLLIYNANTLEEGVPSRFRGSGSGTDFTLTISSLQPEDFATYFCKQAYDVPRTFGGGTKVEIK

SEQ ID NO: 18 - protein, 116 aa, Artificial sequence
  Amino acid sequence of heavy chain variable region of AB126
EVQLVQSGAEVKKPGSSVKVSCKASGYTFTAYWMNWVRQAPGQGLEWIGRIEPYDSETRYNQNFRDRVTLTVDKSSSTAYMELSSLRSEDTAVYYCARGVRIFDYWGQGTTVTVSS

SEQ ID NO: 19 - protein, 107 aa, Artificial sequence
  Amino acid sequence of light chain variable region of AB126
DIQMTQSPSSLSASVGDRVTITCRTSENIDYTLAWYQQKPGKSPKLLIYNANTLEEGVPSRFRGSGSGTHFTLTISSLQPEDFATYFCKQAYDVPRTFGGGTKVEIK

SEQ ID NO: 20 - protein, 116 aa, Artificial sequence
  Amino acid sequence of heavy chain variable region of AB127
EVQLVQSGAEVKKPGSSVKVSCKASGYTFTAYWMNWVKQRPGQCLEWIGRIDPYDSETRYNQNFRDKARLTVDKSSSTAYMELSSLRSEDTAVYYCARGVRIFDYWGQGTTVTVSS

SEQ ID NO: 21 - protein, 107 aa, Artificial sequence
  Amino acid sequence of light chain variable region of AB127
DIQMTQSPSSLSASVGDRVTITCRTSENIDYTLAWYQQKPGKSPKLLIYNANTLEEGVPSRFRGSGSGTHFTLTISSLQPEDFATYFCKQAYDVPRTFGCGTKVEIK

SEQ ID NO: 22 - protein, 116 aa, Artificial sequence
  Amino acid sequence of heavy chain variable region of AB128
EVQLVQSGAEVKKPGSSVKVSCKASGYTFTAYWMNWVKQRPGQGLEWIGRIDPYDSETRYNQNFRDKARLTVDKSSSTAYMELSSLRSEDTAVYYCARGVRIFDYWGQGTTVTVSS

SEQ ID NO: 23 - protein, 107 aa, Artificial sequence; identical to SEQ ID NO: 19
  Amino acid sequence of light chain variable region of AB128

SEQ ID NO: 24 - protein, 17 aa, Artificial sequence
  HCDR2
RIEPYDSETRYNQNFRD

SEQ ID NO: 25 - protein, 7 aa, Artificial sequence
  LCDR2
NANTLEE

SEQ ID NO: 26 - protein, 8 aa, Artificial sequence
  HCDR2
IEPYDSET